(19) United States
(12) Patent Application Publication
(10) Pub. No.: US 2025/0009501 A1
(43) Pub. Date: Jan. 9, 2025

(54) LEAFLET DESIGN FOR A HEART VALVE PROSTHESIS

(71) Applicant: MEDTRONIC, INC., Minneapolis, MN (US)

(72) Inventors: Sara Salehyar, Irvine, CA (US); Justin Goshgarian, Santa Rosa, CA (US); Yogesh Darekar, Irvine, CA (US)

(21) Appl. No.: 18/711,606

(22) PCT Filed: Nov. 23, 2022
(86) PCT No.: PCT/IB2022/061354

§ 371 Date: May 19, 2024

Related U.S. Application Data
(60) Provisional application No. 63/264,482, filed on Nov. 23, 2021.

(51) Int. Cl. A61F 2/24

(52) U.S. Cl. CPC A61F 2/2418; A61F 2230/0004

(57) ABSTRACT

A prosthetic valve component is disclosed for a valve prosthesis. The valve component includes valve leaflets configured to be operably coupled within a frame of the valve prosthesis, wherein each valve leaflet is shaped and sized to achieve a target pinwheeling score PS. The target pinwheeling score correlates to full contact being made between free edges of the valve leaflets, when the valve leaflets are operably coupled within the frame of the valve prosthesis, such that no central opening and little to no overlap occurs between the valve leaflets.

### FIELD

The present technology is generally related to heart valve prostheses implantable via minimally invasive procedures, and in particular is directed to mitral valve prostheses having prosthetic valve leaflets.

### BACKGROUND

The human heart is a four chambered, muscular organ that provides blood circulation through the body during a cardiac cycle. The four main chambers include the right atrium and right ventricle which supplies the pulmonary circulation, and the left atrium and left ventricle which supplies oxygenated blood received from the lungs into systemic circulation. To ensure that blood flows in one direction through the heart, atrioventricular valves (tricuspid and mitral valves) are present between the junctions of the atrium and the ventricles, and semi-lunar valves (pulmonary valve and aortic valve) govern the exits of the ventricles leading to the lungs and the rest of the body. These valves contain leaflets or cusps that open and shut in response to blood pressure changes caused by the contraction and relaxation of the heart chambers. The valve leaflets move apart from each other to open and allow blood to flow downstream of the valve, and coapt to close and prevent backflow or regurgitation in an upstream manner.

Diseases associated with heart valves, such as those caused by damage or a defect, can include stenosis and valvular insufficiency or regurgitation. For example, valvular stenosis causes the valve to become narrowed and hardened which can prevent blood flow to a downstream heart chamber from occurring at the proper flow rate and may cause the heart to work harder to pump the blood through the diseased valve. Valvular insufficiency or regurgitation occurs when the valve does not close completely, allowing blood to flow backwards, thereby causing the heart to be less efficient. A diseased or damaged valve, which can be congenital, age-related, drug-induced, or in some instances, caused by infection, can result in an enlarged, thickened heart that loses elasticity and efficiency. Some symptoms of heart valve diseases can include weakness, shortness of breath, dizziness, fainting, palpitations, anemia and edema, and blood clots which can increase the likelihood of stroke or pulmonary embolism. Symptoms can often be severe enough to be debilitating and/or life threatening.

Heart valve prostheses have been developed for repair and replacement of diseased and/or damaged heart valves. Such heart valve prostheses can be percutaneously delivered and deployed at the site of the diseased heart valve through catheter-based delivery systems. Such heart valve prostheses are delivered in a radially compressed or crimped configuration so that the heart valve prosthesis can be advanced through the patient's vasculature. Once positioned at the treatment site, the heart valve prosthesis is expanded to engage tissue at the diseased heart valve region to, for instance, hold the heart valve prosthesis in position.

While these heart valve prostheses offer minimally invasive methods for heart valve repair and/or replacement, challenges remain when implanting a heart valve prosthesis within a native mitral valve having high ellipticity. Particularly, a lack of coaptation of prosthetic leaflets at a center of a heart valve prosthesis may occur, after deployment within a native mitral valve, due to the ellipticity of the mitral valve annulus and such a lack of central coaptation of the valve leaflets may result in undesirable regurgitation. The present disclosure relates to improvements in a heart valve prosthesis to ensure that valve leaflets of the heart valve prosthesis properly coapt with each other in such a manner as to minimize or entirely avoid central regurgitation, while maintaining the durability of the valve leaflets and low profile of the heart valve prosthesis.

### SUMMARY

The devices and methods of this disclosure generally relate to a heart valve prosthesis that is configured to be implanted within a native heart valve.

In an aspect, which may be combined with any of the other aspects noted herein, the disclosure provides a prosthetic valve component for a valve prosthesis that includes valve leaflets configured to be operably coupled within a frame of the valve prosthesis, wherein each valve leaflet is shaped and sized to achieve a target pinwheeling score PS, the target pinwheeling score correlating to full contact between the valve leaflets along their free edges, when operably coupled within the frame of the valve prosthesis, whereby no central opening and little to no overlap occurs between the valve leaflets.

In another aspect, which may be combined with any of the other aspects noted herein, the disclosure provides a prosthetic valve component for which a pinwheeling score PS is related to a ratio of a free edge length FEL in a closed state of an individual valve leaflet of the valve leaflets to an inner diameter ID of the frame within which the valve leaflets are to be operably coupled, and is calculated by the equation PS=1−5 (|(FEL/ID)−1|), PS=1 when (FEL/ID)<1.

In another aspect, which may be combined with any of the other aspects noted herein, the disclosure provides a prosthetic valve component for which a pinwheeling score is 0.96.

In another aspect, which may be combined with any of the other aspects noted herein, the disclosure provides a prosthetic valve component for which a pinwheeling score is in a range of 0.94 and 0.96.

In another aspect, which may be combined with any of the other aspects noted herein, the disclosure provides a prosthetic valve component in which each valve leaflet is shaped to have an arcuate base edge, a first commissure edge, a second commissure edge, and a free edge that extends between and connects the first and second commissure edges.

In another aspect, which may be combined with any of the other aspects noted herein, the disclosure provides a prosthetic valve component in which a first commissure edge and a second commissure edge of a valve leaflet are angled slightly outward from respective ends of an arcuate base edge of the valve leaflet.

In another aspect, which may be combined with any of the other aspects noted herein, the disclosure provides a prosthetic valve component in which a free edge of the valve leaflet has a first end, where the free edge meets a first commissure edge, and a second end, where the free edge meets the second commissure edge, and wherein a free edge length FEL of the valve leaflet is a distance of the free edge as measured between the first end and the second end thereof.

In another aspect, which may be combined with any of the other aspects noted herein, the disclosure provides a prosthetic valve component in which a free edge of a valve leaflet is slightly raised at a midpoint thereof such that a height of the valve leaflet at the midpoint of the free edge is greater than a height of the valve leaflet at either a first end or a second end of the free edge.

In another aspect, which may be combined with any of the other aspects noted herein, the disclosure provides a prosthetic valve component in which a first height of a valve leaflet at each of a first end and a second end of a free edge thereof gradually increases to a second height of the valve leaflet at a midpoint of the free edge.

In another aspect, which may be combined with any of the other aspects noted herein, the disclosure provides a prosthetic valve component in which a second height of a valve leaflet is 3% to 6.5% greater than a first height of the valve leaflet.

In another aspect, which may be combined with any of the other aspects noted herein, the disclosure provides a prosthetic valve component in which there are three valve leaflets.

In another aspect, which may be combined with any of the other aspects noted herein, the disclosure provides a valve prosthesis that includes a frame and a prosthetic valve component in accordance with an aspect or aspects of the disclosure operably coupled within the frame.

In another aspect, which may be combined with any of the other aspects noted herein, the disclosure provides a valve prosthesis in which a frame includes a valve support element within which valve leaflets of a prosthetic valve component are operably coupled and an inner diameter of the frame is an inner diameter of the valve support element.

In another aspect, which may be combined with any of the other aspects noted herein, the disclosure provides a prosthetic valve component of a valve prosthesis includes three valve leaflets and each valve leaflet is shaped to have a free edge that is slightly raised at a midpoint thereof such that a height of the valve leaflet at the midpoint of the free edge is greater than a height of the valve leaflet at either a first end or a second end of the free edge.

In another aspect, which may be combined with any of the other aspects noted herein, the disclosure provides a prosthetic valve component of a valve prosthesis in which a first height of each valve leaflet at each of a first end and a second end of a free edge gradually increases to a second height of the valve leaflet at a midpoint of the free edge.

In another aspect, which may be combined with any of the other aspects noted herein, the disclosure provides a prosthetic valve component of a valve prosthesis in which a second height of a valve leaflet is 3% to 6.5% greater than a first height of the valve leaflet.

In another aspect, which may be combined with any of the other aspects noted herein, the disclosure provides a prosthetic valve component of a valve prosthesis in which a pinwheeling score is 0.96.

In another aspect, which may be combined with any of the other aspects noted herein, the disclosure provides a prosthetic valve component of a valve prosthesis in which a pinwheeling score is in a range of 0.94 and 0.96.

### BRIEF DESCRIPTION OF DRAWINGS

The foregoing and other features and advantages of the invention will be apparent from the following description of embodiments thereof as illustrated in the accompanying drawings. The accompanying drawings, which are incorporated herein and form a part of the specification, further serve to explain the principles of the invention and to enable a person skilled in the art to make and use the invention. The drawings are not to scale.

FIG. 1 depicts a perspective view of a transcatheter heart valve prosthesis in accordance with an aspect of the disclosure.

FIG. 2 depicts a perspective view of an outflow end of the heart valve prosthesis of FIG. 1 in accordance with an aspect of this disclosure with a prosthetic valve component shown in an open position.

FIG. 3 depicts a perspective view of an inner portion of the heart valve prosthesis of FIG. 1 in accordance with an aspect of the disclosure.

FIG. 4 depicts a bottom or outflow view of the heart valve prosthesis of FIG. 1 in accordance with an aspect of the disclosure.

FIG. 5 depicts a prosthetic valve leaflet in accordance with an aspect of the disclosure for attachment within a heart valve prosthesis of FIG. 1.

### DETAILED DESCRIPTION

Specific embodiments of the present invention are now described with reference to the figures, wherein like reference numbers indicate identical or functionally similar elements. The terms “inflow” and “outflow”, when used in the following description refer to a native vessel, native valve, or a device to be implanted into a native vessel or native valve, such as a heart valve prosthesis, are with reference to the direction of blood flow. Thus, “inflow” refers to positions in an upstream direction with respect to the direction of blood flow and the term “outflow” refers to positions in a downstream direction with respect to the direction of blood flow.

The following detailed description is merely exemplary in nature and is not intended to limit the invention or the application and uses of the invention. Although the description of embodiments hereof is in the context of the treatment of heart valves such as the pulmonary, aortic, mitral, or tricuspid valve, the invention may also be used in other body passageways where it is deemed useful. Furthermore, there is no intention to be bound by any expressed or implied theory presented in the preceding technical field, background, brief summary or the following detailed description.

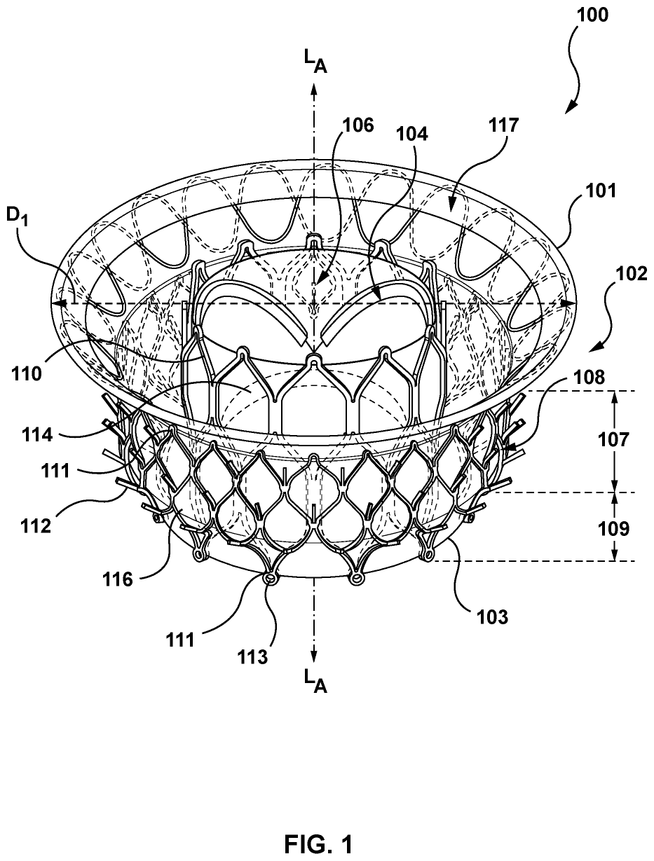

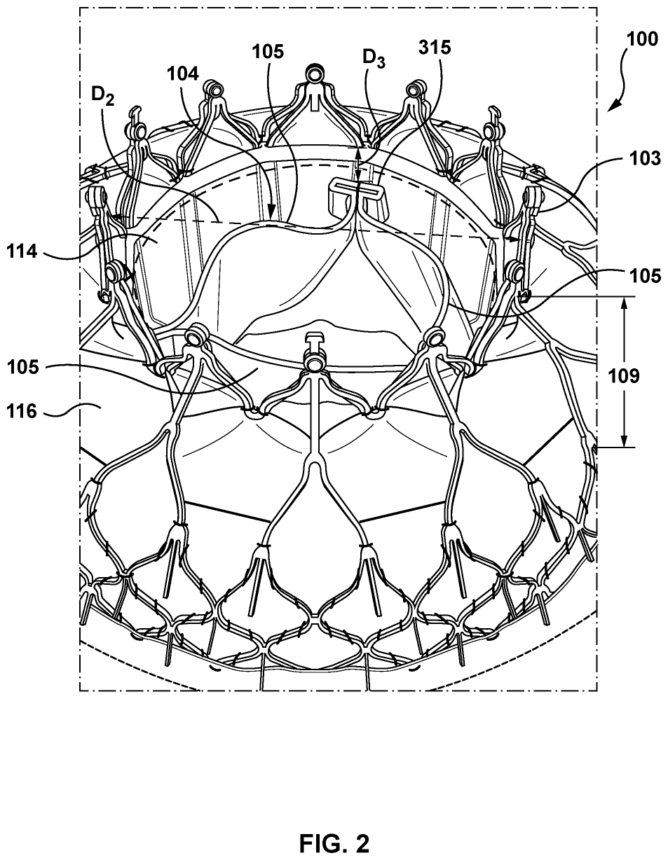

A perspective view of a transcatheter heart valve prosthesis 100 in accordance with an aspect of the disclosure is shown in FIG. 1, with FIG. 2 depicting a perspective view of an outflow end of the heart valve prosthesis 100. The heart valve prosthesis 100 is configured to be compressed into a reduced-diameter delivery configuration within a delivery catheter and to return to an expanded, deployed configuration when delivered/released from the delivery catheter within a native mitral valve. The heart valve prosthesis 100 includes a frame 102 and a prosthetic valve component 104, which is shown in an open position in FIG. 2. The frame 102 has a stent-like structure that is configured to support the prosthetic valve component 104 and to define, along a longitudinal axis LA thereof, a blood flow lumen 106 that substantially extends from an inflow end 101 to an outflow end 103 of the heart valve prosthesis 100.

In some embodiments, the frame 102 includes an outer portion 108 and an inner portion 110. In aspects hereof, the inner portion 110 of the frame 102 may be alternatively referred to as a valve support element, an inner frame, or valve housing, and/or the outer portion 108 of the frame 102 may be alternatively referred to as an anchoring element, a fixation ring, or an outer frame. The inner portion 110 is configured to hold the prosthetic valve component 104, and the outer portion 108, which surrounds the inner portion 110, is configured to secure the heart valve prosthesis 100 to the native tissue of the heart when implanted in vivo. The frame 102 may be considered to have a dual-stent structure, i.e., an inner stent and an outer stent. In some embodiments, without departing from the scope hereof, a frame 102 may be of a single stent structure as to not include an outer stent structure, such as an outer portion 108.

In accordance with aspects hereof, the inner portion 110 and the outer portion 108 of the frame 102 of the heart valve prothesis 100 may be made from any number of suitable biocompatible materials, e.g., stainless steel, nickel titanium alloys such as Nitinol™, cobalt chromium alloys such as MP35N, other alloys such as ELGILOY® (Elgin, Ill.), various polymers, pyrolytic carbon, silicone, polytetrafluoroethylene (PTFE), or any number of other materials or combination of materials. A suitable biocompatible material would be selected such that the heart valve prosthesis 100 may be configured to be compressed into a reduced diameter configuration for transcatheter delivery to a native valve, whereby release from a delivery catheter allows the prosthesis 100 to self-expand, returning to an expanded, deployed configuration. In some embodiments, the self-expansion is accomplished through the use of a shape-memory material such as Nitinol™. The heart valve prosthesis 100 may be processed to have a default or “set” shape that coincides with the deployed configuration. Therefore, once the compressed heart valve prosthesis 100 is delivered and released, the prosthesis 100 will return to the default or “set” deployed configuration.

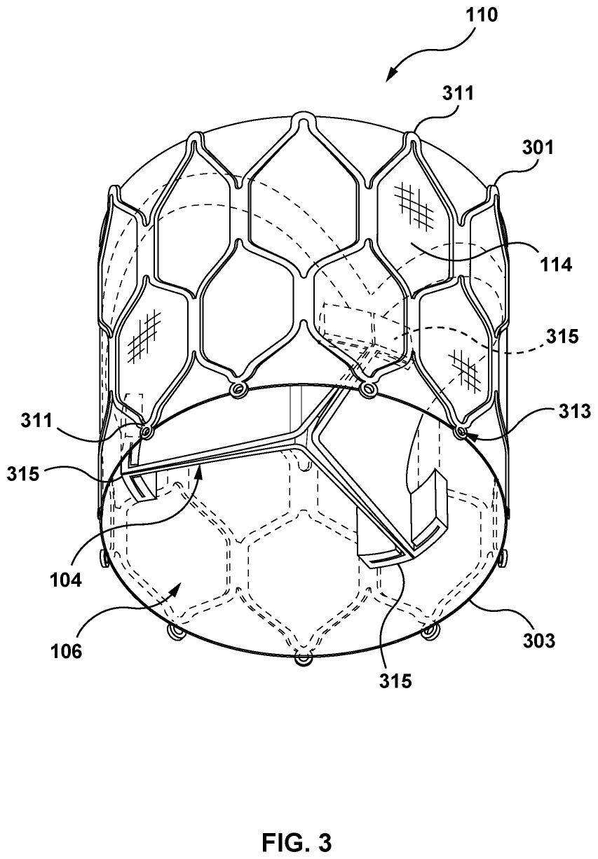

The inner portion 110 is positioned within the outer portion 108 so as to be spaced therefrom, or stated in another way, to be mechanically isolated therefrom. FIG. 3 depicts a perspective view of the inner portion 110 of the heart valve prosthesis 100 in accordance with an aspect of this disclosure and shows the interior of an outflow end 303 of the inner portion 110. The inner portion 110 generally forms a hollow cylindrical shape having a substantially constant inner diameter ID from an inflow end 301 to the outflow end 303 thereof.

In an aspect hereof, a stent-like structure of the inner portion 110 defines a plurality of open cells arranged in a honeycomb pattern. An inner skirt 114, which may take the form of a single piece or multiple pieces of material, is disposed within, and coupled or fused to the inner portion 110 to inhibit blood flow through the open cells of the stent-like structure thereof. Further, each of the inflow end 301 and the outflow end 303 of the inner portion 110 includes a plurality of crowns 311, with the crowns 311 at the outflow end 303 each including an aperture 313 that allows for the inner portion 110 to be coupled to the outer portion 108 as described in detail below.

The outer portion 108 is configured to secure the heart valve prosthesis 100 to the native mitral valve and the surrounding subannular tissue, such as the inward facing-surface of native mitral valve leaflets. The outer portion 108 is positioned around the inner portion 110 and defines the inflow end 101 of the heart valve prosthesis 100, having a first diameter D1, and the outflow end 103 of the heart valve prosthesis 103, having a second diameter D2 that is smaller than the first diameter D1. The outer portion 108 includes a transition segment 109 that tapers from the first diameter D1 to the smaller second diameter D2.

In an aspect hereof, a stent-like structure of the outer portion 108 is defined by a plurality of open cells arranged in a lattice-like pattern within an upstream cylindrical segment 107 thereof, and by Y-shaped struts within the transition segment 109 thereof. An outer skirt 116, which may take the form of a single piece or multiple pieces of material, is disposed within, and coupled or fused to the outer portion 108 to inhibit blood flow through the open cells and Y-shaped struts of the stent-like structure thereof. Further, each of the inflow end 101 and the outflow end 103 of the outer portion 108 includes a plurality of crowns 111, with the crowns 111 at the outflow end 103 each including an aperture 113. The apertures 313 of the plurality of crowns 311 at the outflow end 303 of the inner portion 110 align with respective apertures 113 of the plurality of crowns 111 at the outflow end 103 of the outer portion 108 in order to permit the coupling together of the inner portion 110 and the outer portion 108 by rivets, welding, or other methods known in the art.

In aspects hereof, the inner skirt 114 lining the inner portion 110 and the outer skirt 116 lining the outer portion 108 may be formed of a low-porosity woven fabric, such as polyester, Dacron fabric, or PTFE. In further aspect, the inner and outer skirts 114, 116 may be a knit polyester, such as a polyester or PTFE knit, which can be used when it is desired to provide a medium for tissue ingrowth and the ability for the fabric to stretch to conform to a curved surface. Polyester velour fabrics may alternatively be used, such as when it is desired to provide a medium for tissue ingrowth on one side and a smooth surface on the other side. These and other appropriate cardiovascular fabrics are commercially available from Bard Peripheral Vascular, Inc. of Tempe, Ariz., for example. In alternative aspects hereof, the inner and outer skirts 114, 116 may be formed of a natural or biological material such as pericardium or another membranous tissue such as intestinal submucosa. Further, it is envisioned that the inner skirt 114 and the outer skirt 116 may be made of the same or different materials, for example, the inner skirt 114 may be made from a PTFE knit, while the outer skirt 116 is made of a woven polyester. It is further envisioned, that in some embodiments, it may be beneficial to have skirts of varying thicknesses, such as, an inner skirt 118 that is thicker than an outer skirt 116.

An outer surface of the outer portion 108, when the heart valve prosthesis 100 is in an expanded state, is configured to be disposed against the native tissue of the mitral valve for securing and anchoring the heart valve prosthesis 100 at the implantation site. In order to provide this anchoring function, the outer portion 108 may further include a plurality of prongs 112 that extend radially from the outer surface of the outer portion 108 and are configured to engage with the native tissue. In aspects hereof, the outer portion 108 may deform upon implantation within a native mitral valve annulus, and/or expand and contract in response to movement of the native tissue during diastole and systole, while remaining spaced from the inner portion 110, which thereby permits the inner portion 110 to remain relatively still and substantially undeformed. Whereas the inner portion 110 is, therefore, substantially isolated from external forces to thereby substantially maintains its cylindrical shape after implantation, when the heart valve prosthesis 100 is implanted within a mitral valve annulus with high ellipticity, the inner portion 110 may undergo some flexing or minor deformation that can adversely affect coaptation of valve leaflets 105 of the prosthetic valve component 104, which is addressed by aspects hereof as described below.

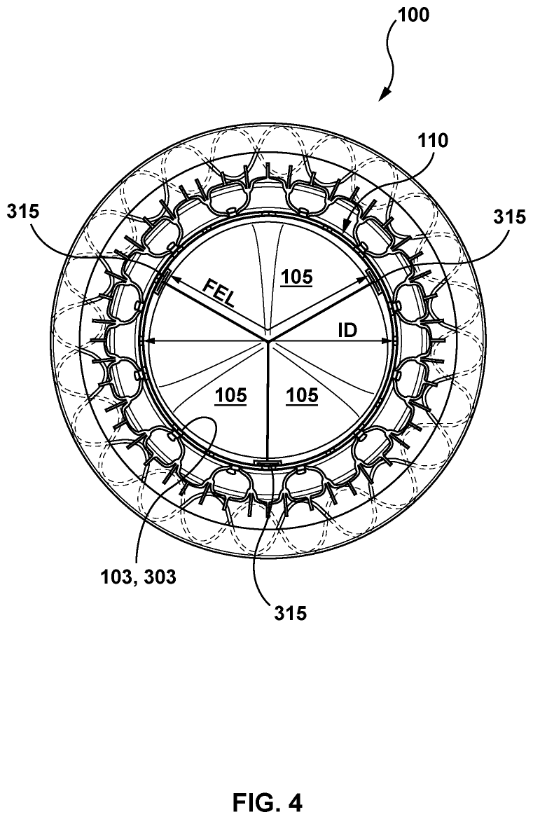

FIGS. 1-4 illustrate an exemplary prosthetic valve component 104 having three valve leaflets 105. Without departing from the scope of the disclosure, a prosthetic valve component in aspects hereof may include fewer or more than three valve leaflets as may be appropriate for other applications. The prosthetic valve component 104 of the heart valve prosthesis 100 is configured to regulating blood flow through the heart valve prosthesis 100 via the valve leaflets 105. When deployed in situ within a native mitral valve, the prosthetic valve component 104 in a closed state (as shown in FIGS. 2 and 4) blocks blood flow in one direction to regulate blood flow through the blood flow lumen 106 of the inner portion 110. More particularly, the valve leaflets 105 are configured to coapt with each other during systole, without any central opening or space therebetween to prevent central regurgitation, and to open during diastole to allow blood flow through the heart valve prosthesis 100. Preferably, when in the closed state, the valve leaflets 105 are in contact along their free edges with little to no overlap or redundancy therebetween, as any overlap or redundancy may result in abrasion of the free edges of the valve leaflets 105 due to the free edges rubbing against each other when the valve leaflets 105 open and close under normal operating conditions.

In aspects in accordance herewith, valve leaflets 105 may be shaped and sized to achieve a preferred or target pinwheeling score PS. A pinwheeling score PS is a ratio related to a free edge length FEL of a valve leaflet in the closed state to an inner diameter ID of a frame within which the valve leaflet is operably coupled, and may be calculated by the equation as follows:

$$PS = 1 - 5 ( \vert ( FEL / ID ) - 1 \vert ) , PS = 1 when ( FEL / ID ) < 1.$$

A preferred or target pinwheeling score PS of a valve leaflet 105 correlates to proper coaptation of the valve leaflets 105 when the valve leaflets 105 are positioned and secured within the inner portion 110 of the heart valve prosthesis 100 such that there is no central opening and little to no overlap or redundancy present between the valve leaflets 105. By proper coaptation it is meant that the valve leaflets 105 make full contact with each other along substantially the entire length of their free edges 105. With reference to FIG. 4, a pinwheeling score PS of a valve leaflet 105 is calculated according to the equation as noted above, which is related to the ratio of a free edge length FEL of the valve leaflet 105 in the closed state to the inner diameter ID of the inner portion 110 of the frame 102. In an aspect hereof, it has been found that a target pinwheel score PS in a range of 0.94 to 0.96 beneficially resulted in full contact of the valve leaflets 105 along their free edges with no central opening and little to no overlap or redundancy occurring between the valve leaflets 105. For example, when a free edge length of a valve leaflet in a closed state is 27.52 mm, i.e., FEL=27.52 and an inner diameter of a frame holding the valve leaflet is 27.3 mm, i.e., ID=27.3, than a target pinwheel score is achieve of PS=0.959.

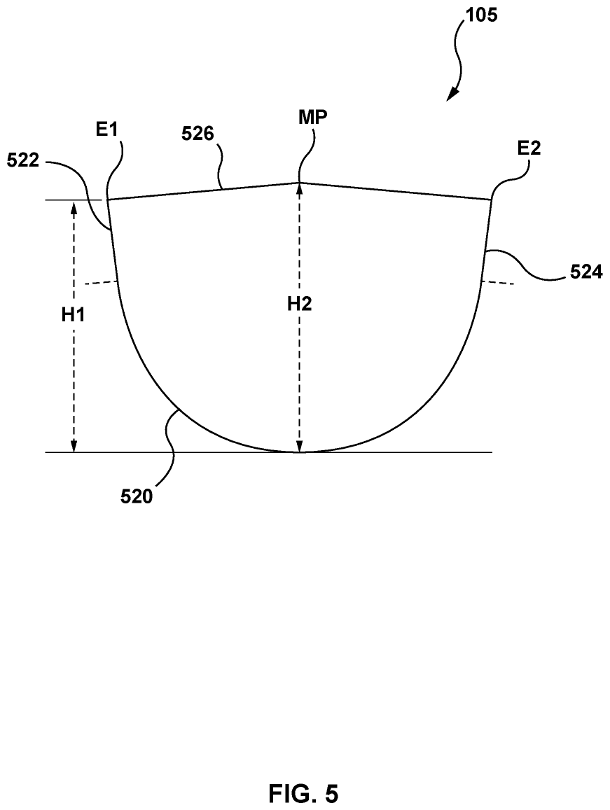

FIG. 5 depicts a prosthetic valve leaflet 105 in accordance with an aspect of the disclosure. The valve leaflet 105 is configured to provide a suitable pinwheel score PS when attached, with remaining valve leaflets 105, within a heart valve prosthesis 100 of FIG. 1. The valve leaflet 105 has an arcuate base edge 520 that is configured to be attached or fused to the inner portion 104, forming what is known as a margin of attachment (MOA) therewith. In aspects hereof, the curved MOA of the valve leaflet 105 may be created or formed along the arcuate base edge 520, for example, by using sutures or a suitable biocompatible adhesive.

The valve leaflet 105 also has a first commissure edge 522 and a second commissure edge 524 defined along opposing segments of its lateral edges, as shown in FIG. 5, that are angled slightly outward from respective ends of the arcuate base edge 520. The first and second commissure edges 522, 524 are segments of the valve leaflet 105 configured for attaching to, and forming a commissure 315 with, a corresponding commissure edge of adjacent valve leaflets 105. In an embodiment, with reference to FIGS. 2 and 3, commissures 315 of the valve leaflets 105 are disposed at the outflow end 303 of the inner portion 110 to be spaced inward of an edge of the inner skirt 114 by a distance D3. The distance D3 may be any distance that is suitable to avoid pinching, ripping, or other type of damage to the valve leaflets 105, by for instance exposed outflow portions of the frame 102, when the heart valve prosthesis 100 is compressed for delivery.

The valve leaflet 105 also has a free edge 526 that extends between, and connects, the opposing first and second commissure edges 522, 524. Further, the free edge 526 has a first end E1 where it meets the first commissure edge 522 and a second end E2 where it meets the second commissure edge 524. The free edge 526 of the valve leaflet 105 is configured to have a free edge length FEL for providing a pinwheeling score PS that correlates to proper coaptation of the valve leaflets 105 when attached within the inner portion 110. In an aspect hereof, a free edge length FEL is a distance of the free edge 526 as measured between the first end E1 and the second end E2 thereof. In order to achieve a suitable free edge length FEL, the free edge 526 of the valve leaflet 105 is not straight but instead slightly raised at a midpoint MP thereof as to have a greater height at the midpoint MP than a height at either end E1, E2. Stated another way, a height H1 of the valve leaflet 105 at each end E1, E2 of the free edge 526 gradually increases to a height H2 of the valve leaflet 105 at the midpoint MP of the free edge 526 thereby increasing the free edge length FEL over an otherwise straight and even free edge. In aspects in accordance herewith, the height (H2) of the valve leaflet 105 at the midpoint MP of the free edge 526 is 3% to 6.5% greater than the height (H1) of the valve leaflet 105 at each end E1, E2 of the free edge 526.

In embodiments hereof, the benefit of using a pinwheel score PS for designing a valve leaflet 105 of a prosthetic valve component 104 is not limited for use in a heart valve prosthesis 100 having the particular features disclosed above but instead may be adapted for use in designing valve leaflets for other types and sizes of valve prosthesis as would be recognized by one of ordinary skill in the art upon considering this disclosure.

In embodiments hereof, prosthetic valve leaflets 105 may be formed of various flexible materials including, but not limited to, natural pericardial material such as tissue from bovine, equine or porcine origins, or synthetic materials such as polytetrafluoroethylene (PTFE), DACRON® polyester, pyrolytic carbon, or other biocompatible materials. With certain prosthetic leaflet materials, it may be desirable to coat one or both sides of the replacement valve leaflet with a material that will prevent or minimize overgrowth. It is further desirable that the prosthetic leaflet material is durable and not subject to failure due to stretching, deforming, or fatigue.

In embodiments hereof, as shown in FIG. 1, the heart valve prosthesis 100 may further include a brim or pre-shaped wire element 117 that extends outwardly from the inflow end 101 thereof. The brim 117 may be formed from sinusoidal wire forms that are attached and hinged to the outer portion 108 by a suitable biocompatible low-profile fabric used in bioprosthetic implants to promote bio-integration, such as woven polyethylene terephthalate (PET) fabric. The brim element 117 may act as an atrial retainer, if present, and to serve such a function the brim element 117 may be configured to engage tissue above a native annulus, such as a supra-annular surface or some other tissue in the left atrium, to thereby inhibit downstream migration of the heart valve prosthesis 100, for e.g., during atrial systole, as well as mitigate any leakage through any gaps between native tissue and the frame 102.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

1. A valve prosthesis comprising:
a frame; and
a prosthetic valve component comprising valve leaflets operably coupled within the frame, wherein each valve leaflet is shaped and sized to achieve a target pinwheeling score PS, the target pinwheeling score correlating to full contact between the valve leaflets along free edges of the valve leaflets, when operably coupled within the frame of the valve prosthesis and in a closed state,
wherein the pinwheeling score PS is related to a ratio of a free edge length FEL in the closed state of an individual valve leaflet of the valve leaflets to an inner diameter ID of the frame within which the valve leaflets are operably coupled, and is calculated by the equation
$$PS = 1 - 5 ( \vert ( FEL / ID ) - 1 \vert ) , PS = 1 when ( FEL / ID ) < 1 ,$$
and
wherein the target pinwheeling score is in a range of 0.94 and 0.96.

2. The valve prosthesis of claim 1, wherein the pinwheeling score is 0.96.

3. The valve prosthesis of claim 1, wherein each valve leaflet is shaped to have an arcuate base edge, a first commissure edge, a second commissure edge, and a free edge that extends between and connects the first and second commissure edges.

4. The valve prosthesis of claim 3, wherein the first commissure edge and the second commissure edge of the valve leaflet are angled slightly outward from respective ends of the arcuate base edge of the valve leaflet.

5. The valve prosthesis of claim 3, wherein the free edge of the valve leaflet has a first end where the free edge meets the first commissure edge, and a second end where the free edge meets the second commissure edge, and wherein the free edge length FEL of the valve leaflet is a distance of the free edge as measured between the first end and the second end thereof.

6. The valve prosthesis of claim 5, wherein the free edge of the valve leaflet is slightly raised at a midpoint thereof such that a height the valve leaflet at the midpoint of the free edge is greater than a height of the valve leaflet at either the first end or the second end of the free edge.

7. The valve prosthesis of claim 5, wherein a first height of the valve leaflet at each of the first end and the second end of the free edge gradually increases to a second height of the valve leaflet at the midpoint of the free edge.

8. The valve prosthesis of claim 7, wherein the second height of the valve leaflet is 3% to 6.5% greater than the first height of the valve leaflet.

9. The valve prosthesis of claim 1, wherein the valve leaflets are three valve leaflets.

10. (canceled)

11. The valve prosthesis of claim 1, wherein the frame includes a valve support element within which the valve leaflets of the prosthetic valve component are operably coupled, and the inner diameter ID of the frame is an inner diameter of the valve support element.

12-16. (canceled)

17. The valve prosthesis of claim 11, wherein the frame further comprises an outer frame surrounding the valve support element and operably coupled to the valve support element.

18. The valve prosthesis of claim 17, wherein the frame further comprises a brim extending outwardly from an inflow end of the outer frame.

19. The valve prosthesis of claim 1, wherein the target pinwheeling score enables full contact along the free edges of the valve leaflet in the closed with no central opening and little to no overlap between the valve leaflets.

20. The valve prosthesis of claim 1, wherein the free edge length FEL in the closed state is about 27.52 mm and the inner diameter ID of the frame is about 27.3 mm.
